(19) United States
(12) Patent Application Publication
(10) Pub. No.: US 2023/0248808 A1
(43) Pub. Date: Aug. 10, 2023

(54) Joint Health Composition and Use Thereof in Healthy Mammals

(71) Applicant: Lonza Greenwood LLC, Greenwood, SC (US)

(72) Inventors: Vijaya Juturu, North Bruswick, NJ (US); Shane Durkee, Morristown, NJ (US)

(21) Appl. No.: 17/791,324

(22) PCT Filed: Jan. 27, 2021
(86) PCT No.: PCT/US2021/015171

§ 371 Date: Jul. 7, 2022

Related U.S. Application Data
(60) Provisional application No. 62/967,169, filed on Jan. 29, 2020.

(51) Int. Cl. A61K 38/39; A23L 33/17; A61K 38/44; C07K 14/78; C12N 9/02

(52) U.S. Cl. CPC A61K 38/39; A23L 33/17; A61K 38/446; C07K 14/78; C12N 9/0089; C12Y 115/01001

(57) ABSTRACT

The present disclosure is directed to a method and composition for improving at least one of joint pain, muscle pain, or fitness in healthy mammals. The method and composition include administering a supplement that includes a type II collagen and a SOD enriched supplement to a healthy mammal that is undergoing or has undergone physical activity or intensive physical activity.

### BACKGROUND

In recent years, the use of collagen to treat various conditions has become exceedingly popular. Collagen is a protein that can be found in muscles, bones, skin, blood vessels, and in other parts of the body. There are various different types of collagen depending upon its function and form. For instance, Type I collagen, the most abundant collagen, is made of fibers found in tendons, ligaments, organs and skin. Type II collagen, on the other hand, primarily helps build cartilage, a major structural entity that sits on the surfaces of those bones which comprise articulating joints. Type III collagen is a major component of the extracellular matrix that makes up organs and skin. Type III collagen also forms blood vessels and tissue within the heart. Type IV collagen is found primarily in the skin as sheet-like structures in the cutaneous basal lamina. Furthermore, Collagen peptides are portions of one or more of the a strands of any type of collagen formed through enzymatic hydrolysis of collagen. Collagen peptides are often used in beverages and food products, as they are water-soluble and non-gelling.

Collagen has also been found to effectively treat arthritis and other joint pain. For example, U.S. Pat. No. 9,066,926 discloses a method of reducing exercise-induced joint pain in mammals by administering to a mammal Type II collagen. This patent also discloses the mechanism of action through which this ingredient operates: oral tolerance. This putative mechanism entails the stimulation of T regulatory cells (Treg), located in gut associated lymphatic tissue, to specifically recognize antigenic determinants (epitopes) on the native collagen protein. Once induced, the Tregs exit the gut area and migrate to the joint space where they stimulate chondrocytes to lay down new Type II collagen thereby enhancing the structural integrity and flexibility of the articulating joint. One such example, for which clinical data has been published is the knee. The '926 patent is incorporated herein by reference.

While both rheumatoid and osteoarthritis are inflammatory conditions not all joint pain is associated with an inflammatory condition and not all pain related to exercise in healthy mammals is related to joint pain. Such muscle and joint pain is frequently induced by exercise or other mechanical stressors, and not disease related factors, and thus, there remains a desire for therapies capable of preventing and treating such pain in humans and other mammals. Thus, although collagen can offer various advantages when administered to a human or animal, a need exists for a composition and method that can increase the effectiveness of collagen and/or work in conjunction with collagen to provide synergistic effects such improved efficacy or faster onset of action to improve the fitness, joint health, muscle health, or combinations thereof, in a healthy mammal.

### SUMMARY OF THE INVENTION

The present disclosure is generally directed to a method of improving one or more of fitness, joint pain, or muscle pain in healthy mammals that have undergone or are undergoing physical activity. The method includes administering a composition that includes a collagen composition and a SOD enriched supplement to a healthy mammal in an amount sufficient to lessen joint pain, lessen muscle pain, or improve fitness of the mammal. Furthermore, the collagen composition includes a type II collagen, where the type II collagen is present in the composition in an amount from about 1 mg to about 1000 mg.

In one aspect, the SOD enriched supplement and the collagen composition are present in the composition at a ratio of type II collagen to SOD enriched supplement of about 100:1 to about 1:100.

Moreover, in an aspect the SOD enriched supplement comprises T. chuii. Additionally or alternatively, in one aspect, the SOD enriched supplement is a biomass of T. chuii, an extract of T. chuii, or a combination thereof. In yet a further aspect, the SOD enriched supplement is present in the composition in an amount of about 1 mg to about 100 mg. Furthermore, in an aspect, the SOD enriched supplement has a SOD activity in the composition of about 500 IU or greater. In another aspect, an oxygen radical absorbance capacity (ORAC) of the SOD enriched supplement is about 100 μmol TE/g or greater. In yet a further aspect, the oxygen radical absorbance capacity (ORAC) of the SOD enriched supplement in the composition is about 2.5 μmol TE or greater.

Furthermore, in one aspect, the composition is administered daily. Additionally or alternatively, the composition further includes at least one of a vitamin, a mineral, an essential fatty acid, or an amino acid. In an aspect, the SOD enriched supplement includes vitamin E, vitamin C, or a combination thereof. In a further aspect, the SOD enriched supplement includes potassium, magnesium, calcium, or combinations thereof.

In another aspect, the lessening of joint pain, muscle pain, or improvement in fitness is evidenced by an improvement in fitness of about 10% or greater than a mammal not being administered the composition, as measured by time to exhaustion. In one aspect, the lessening of joint pain, muscle pain, or improvement in fitness is evidenced by a lessening of cholesterol of about 10% greater than a mammal not being administered the composition. Moreover, in an aspect, the lessening of joint pain, muscle pain, or improvement in fitness is evidenced by the mammal exhibiting about 10% less oxidative stress markers than a mammal not being administered the composition. Additionally or alternatively, the lessening of joint pain, muscle pain, or improvement in fitness is evidenced by the mammal exhibiting cartilage breakdown indicators of about 5% less than a mammal not being administered the composition.

In one aspect, the healthy mammal is not suffering from arthritis. Furthermore, in one aspect, the physical activity is intense physical activity. In another aspect, the mammal has an age of about 55% or less of its expected life span. In another aspect, the mammal has an age of about 60% or more of its expected life span.

In yet a further aspect, the type II collagen is present in the composition in an amount of about 5 mg to about 500 mg. Additionally or alternatively, the type II collagen is present in the composition in an amount of about 15 mg to about 200 mg. In another aspect, the type II collagen is present in the composition in an amount of about 20 mg to about 100 mg. Furthermore, in an aspect, the type II collagen includes native type II collagen and undenatured type II collagen. Moreover, in an aspect, the undenatured type II collagen is present in the composition in an amount of about 0.25 mg to about 100 mg.

The present disclosure is also generally directed to a composition for improving one or more of fitness, joint pain, or muscle pain in healthy mammals that have undergone or are undergoing physical activity. The composition includes a collagen composition that contains a type II collagen. The collagen composition has a total oxygen radical absorbance capacity (ORAC) of about 200 μmol TE/g or greater, and the type II collagen is present in the composition in an amount of from about 1 mg to about 1000 mg. The composition also includes a SOD enriched supplement having a total ORAC of about 100 μmol TE/g or greater.

In one aspect, the type II collagen includes native type II collagen and undenatured type II collagen. In a further aspect, the undenatured type II collagen is present in the composition in an amount of about 0.25 mg to about 100 mg.

In yet another aspect, the composition is formulated into one or more delivery forms. In one aspect, the composition is coated on a core, contained in a lipid multiparticulate, or contained in an oil-in-water emulsion. Furthermore, in an aspect, the one or more delivery forms is suitable for incorporation into a capsule, a beverage, a food product, or combinations thereof.

Furthermore, in one aspect, the SOD enriched supplement and the type II collagen are present in the composition at a ratio of type II collagen to SOD enriched supplement of about 100:1 to about 1:100. Additionally or alternatively, the SOD enriched supplement comprises T. chuii. In another aspect, the SOD enriched supplement is a biomass of T. chuii, an extract of T. chuii, or a combination thereof. In one aspect, the SOD enriched supplement is present in the composition in an amount of about 1 mg to about 100 mg. Moreover, in an aspect, the SOD enriched supplement has a SOD activity in the composition of about 500 IU or greater.

### BRIEF DESCRIPTION OF THE FIGURES

FIG. 1 shows the ratio of compositions according to the present disclosure to a control of several muscle gene proteins;

FIG. 2 shows the ratio of compositions according to the present disclosure to a control of muscle gene proteins; and

FIG. 3 shows the ratio of compositions according to the present disclosure to a control of muscle gene proteins.

### DEFINITIONS

As used herein, the terms “about,” “approximately,” or “generally,” when used to modify a value, indicates that the value can be raised or lowered by 10% and remain within the disclosed aspect.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “nutraceutical” and “ingredient” refers to any compound added to a dietary source (e.g., a fortified food or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved resilience and recovery and can include without limitation canine, equine, feline, bovine, ovine, human, or porcine mammals.

As used herein, “healthy” refers to the absence of illness or injury.

The term “physical activity” means activity that lasts about 10 minutes or more, such as about 25 minutes or more, such as about 30 minutes or more, such as at least about 45 minutes or more, and where the heart rate of the mammal reaches about 30% to about 85% of its maximum heart rate, such as about 40% to about 80%, such as about 50% to about 75% of the maximum heart rate of the mammal.

The term “intensive physical activity” means activity that lasts about 20 minutes or more, such as about 25 minutes or more, such as about 30 minutes or more, such as at least about 45 minutes or more, and where the heart rate of the mammal reaches about 50% to about 99% of its maximum heart rate, such as about 55% to about 95%, such as about 60% to about 90%, such as about 705 to about 85% of the maximum heart rate of the mammal.

Unless otherwise noted, “collagen” as used herein refers to all forms of collagen, either with or without denaturation, without or without salts or stabilizing agents, and fibrillar and non-fibrillar types of collagen not limited to fibril associated collagens with interrupted triple helices (FACIT, Type IX, XII, XIV, XIX, XXI), including short chain collagen (generally Types VII and X), basement membrane (Type IV), Multiplexin (multiple triple helix domains with interruptions (Type XV, XVIII), and other types of collagen (Types VI, VII).

As used herein, “Delivery” refers to approaches, formulations, technologies, and systems for transporting a compound in the body, including the use of nanoparticles, as needed to achieve the compounds desired therapeutic effect and/or nutraceutical effect, as a standalone compound or in combination. The targeted delivery may be controlled release, short acting, and/or long lasting release, not limited to the following:
Immediate release: Coni-Snap® Gelatin, Vcaps® Plus, Plantcaps®, Vcaps® Gen C;
Modified release: Vcaps® Enteric, DRcaps™, Coni-Snap® Gelatin and Conk Snap® Gelatin-PEG, Vcaps® and Vcaps® Plus;
PCcaps®, DBcaps®, Colorista®, End to End: Coni-Snap® Sprinkle, Press-Fit®, Xpress-Fit®, Licaps®;

Furthermore, in one or more aspects, delivery may also include bioavailability enhancing technologies not limited to:
Liquid filled hard capsules: CFS1200™, CFS1500™, LEMS®70
Micronization and containment equipment; and

Technologies for clinical development and small batches, not limited to:
Powder micro-dosing systems: Xcelolab®, Xcelodose® 120S, Scelodose® 600S;
Hard capsule filing machines: Ultra III™.

### Abbreviations

RA=rheumatoid arthritis; OA=osteoarthritis; ECM=extracellular matrix; TNF-α=tumor necrosis factor-alpha; IL-1β=interleukin-1 beta; IL-6=interleukin-6; IL-4=interleukin 4; IL-10=interleukin-10; MMP=matrix metalloproteinase; NF-κB=nuclear factor-kappa-light-chain-enhancer of activated B cells; MAPK=mitogen activated protein kinase; ERK=extracellular receptor kinase; NO=nitric oxide; TGF-β=transforming growth factor-beta; CIA=collagen induced arthritis; KOOS=knee injury and osteoarthritis outcome score; ROM=range of motion; MRI=magnetic resonance imaging; GALT=gut associated lymphatic tissue; QoL=quality of life; MIP-1β=macrophage inflammatory protein-1 beta; IP-10=interferon gamma-induced protein 10; T H=T helper cell; WOMAC=western Ontario and McMaster universities osteoarthritis index; ACR=American College of Rheumatology.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to a method and composition for improving at least one of the fitness, joint health, muscle heath, bone health, and cartilage health of a healthy mammal during or after physical activity or intensive physical activity. Particularly, the present disclosure has found that administering a composition that includes a collagen composition including a type II collagen, alone or in combination with another collagen source, and a superoxide dismutase (SOD) enriched supplement, unexpectedly improves the fitness of a mammal, measured as the time to exhaustion or an increase in strength, reduces joint and/or muscle pain during and after intensive exercise and or physical activity in a healthy mammal, lengthens the period of strenuous exercise in a healthy mammal before joint pain, muscle pain, pain in tendons, and/or cartilage degradation is experienced, or a combination thereof, and may actually improve bone regrowth. The administration of a composition containing type II collagen composition and a SOD enriched supplement also serves to speed recovery from exercise-induced pain in healthy mammals and can reduce inflammation, swelling, and muscle and cartilage breakdown associate with physical activity and intensive physical activity, such as inflammation, swelling, and pain that may be due to oxidative stress. These results are surprising in light of the absence of any autoimmune condition and in light of the fact that exercise-induced pain has generally been considered to be the result of a physical stressor rather than an inflammatory process such as rheumatoid or osteo arthritis. This finding was further surprising, as, while antioxidants have been administered to aging populations that suffer from diseases either due to, or worsened by declines in antioxidant production, there has thus far been no motivation to administer such an antioxidant composition to a healthy, or even trained mammal, that is not experiencing disease-related decline.

In one aspect, for instance, the composition of the present disclosure is particularly formulated to improve joint health, muscle health, cartilage heath, bone health, or combinations thereof. For instance, the composition can be used to treat non-arthritic joint pain, joint discomfort in healthy mammals, lack of joint flexibility in healthy mammals, muscle soreness in healthy mammals, or lack of fitness in healthy mammals. In addition, the supplement of the present disclosure can improve immune health, bone health, or brain health, and may also improve triglyceride and/or cholesterol levels in the healthy mammal.

In general, the supplement of the present disclosure contains a collagen composition. The collagen composition may include one or more of any collagen as defined above, and/or, in one aspect, may include one or more of Type I collagen, Type II collagen, Type III collagen, Type IV collagen, or collagen peptides, or a mixture thereof. In one aspect, the collagen composition contains Type II collagen alone or in combination with one or more of Type I collagen, Type III collagen, Type IV collagen, or collagen peptides. In one aspect, the collagen composition may include a mixture of type II collagen (sometimes referred to as native type II collagen) and undenatured type II collagen. Additionally or alternatively, the collagen composition may include a mixture of native type II collagen and undenatured type II collagen, in addition to a further collagen, such as Type I, Type III, Type IV, or collagen peptides.

Nonetheless in one aspect, the collagen composition includes an undenatured type II collagen, alone or in combination with native type II collagen. Particularly, the present disclosure has found that the SOD enriched supplement and a collagen composition including native and/or undenatured type II collagen, when combined, can increase the effectiveness of the resulting composition. In addition, or alternatively, the combination of a collagen composition including native and/or undenatured type II collagen with a SOD enriched supplement can improve the amount of time needed to recover from muscle pain, joint pain, joint stiffness, as well as other exercise induced or exacerbated pains, including pain related to oxidative stress.

As indicated above, the composition contains a collagen composition, particularly a Type II collagen composition such as an undenatured Type II collagen composition. Type II collagen for use in the present disclosure can be obtained from any suitable source. For instance, the collagen can be derived from a variety of mammalian sources, avian sources, or can be obtained from various fish species or a combination thereof. For instance, the collagen can be obtained from salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II® from InterHealth Nutraceuticals. UC-II® is a natural ingredient that contains a glycosylated, undenatured Type II collagen. The collagen composition can also comprise a hydrolyzed collagen. The collagen composition can also comprise a pure protein or active peptide fragments. In one embodiment, the collagen composition can be free of any bone or bone material. In other embodiments, the collagen composition can be free of any transforming growth factors (TGFs), bone morphogenetic proteins (BMPs), or both. In still another embodiment, the collagen composition comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the type II collagen in the tissue and formed into doses containing therapeutically effective levels of undenatured type II collagen, said levels being generally in the amount of at least about 0.01 gram and preferably from about 0.02 to about 0.5 grams of animal tissue in a dose. Being a natural product some variation from sample to sample is to be expected. These variations can be minimized by blending after comminution. The blending can be aided by analytical techniques which allow the measurement of the amount of undenatured type II collagen and other constituents.

In one aspect, the collagen composition is present in the composition in an amount from about 1 milligram to about 5000 milligrams. For instance, the collagen composition can be present in the composition in an amount greater than about 5 milligrams, such as in an amount greater than about 10 milligrams, such as in an amount greater than about 15 milligrams, such as in an amount greater than about 20 milligrams, such as in an amount greater than about 25 milligrams, such as in an amount greater than about 30 milligrams. The total amount of collagen composition present in the composition is generally be less than about 1000 milligrams, such as less than about 900 milligrams, such as less than about 800 milligrams, such as less than about 700 milligrams, such as less than about 600 milligrams, such as less than about 500 milligrams, such as less than about 400 milligrams, such as less than about 300 milligrams, such as less than about 200 milligrams, such as less than about 100 milligrams, such as less than about 80 milligrams, such as less than about 70 milligrams, such as less than about 60 milligrams, or any ranges or values therebetween. Additionally or alternatively, the collagen composition may be present in the composition in an amount of about 30% to about 90% by weight, such as about 35% to about 85%, such as about 40% to about 80%, such as about 45% to about 75%, such as about 50% to about 70%, such as about 55% to about 65%, or any ranges or values therebetween. Furthermore, it should be understood that, in one aspect, the collagen composition may be a type II collagen composition, where substantially all of the collagen in the collagen composition is type II collagen.

In one aspect, undenatured type II collagen may form all, or substantially all, of the total type II collagen in the collagen composition, and therefore, may be present in the above discussed amounts. However, in one aspect, undenatured type II collagen may account for about 1% to about 95% of the total type II collagen and/or collagen composition, such as about 2.5% to about 75%, such as about 5% to about 50%, such as about 10% to about 40% of the total type II collagen or total collagen composition, or any ranges or values therebetween. Therefore, in one aspect, undenatured type II collagen may be present in the composition in an amount of 0.1 mg to about 100 mg, such as about 0.5 mg to about 75 mg, such as about 0.75 mg to about 50 mg, such as about 1 mg to about 30 mg, or any ranges or values therebetween.

Furthermore, in one aspect, the collagen composition may further include a preservative salt, such as potassium chloride. Thus, in one aspect, the total amounts of collagen composition discussed above may include type II collagen and/or undenatured type II collagen, alone or in combination with a further collagen, a preservative salt, or combinations thereof. In such as aspect, the total type II collagen, including native and undenatured type II collagen, may account for about 1% to about 99% of the collagen composition, such as about 2.5% to about 90%, such as about 5% to about 80%, such as about 7.5% to about 70%, such as about 10% to about 60%, such as about 15% to about 50%, such as about 20% to about 35%, or any ranges or values therebetween. Thus, in one aspect, the total amount of type II collagen, including native and undenatured type II collagen in the collagen composition may be from about 1 mg to about 1000 mg, such as about 2.5 mg to about 500 mg, such as about 5 mg to about 250 mg, such as about 7.5 mg to about 100 mg, such as about 10 mg to about 40 mg, or any ranges or values therebetween. Of course, in one aspect, no preservative salt is used.

Furthermore, in one aspect, when the type II collagen includes undenatured type II collagen, the undenatured type II collagen may have a large oxygen radical absorbance capacity (ORAC), as measured according to ORAC 6.0. Particularly, ORAC tests measure antioxidant scavenging activity against oxygen radicals that are known to be involved in the pathogenesis of aging and common disease, and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as μmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2,2′ azobis(2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosyndnonimine hydrochloride. The ORAC assay against superoxide measures the antioxidant capacity of the sample to protect hydroethidine from damage by a superoxide which is generated from xanthine oxidase. The ORAC assay against singlet oxygen measures the antioxidant capacity of the sample to protect hydroethidine from damage by single oxygen which is generated from a reaction between lithium molybdate and hydrogen peroxide. Finally, the ORAC assay against hypochlorite measures the antioxidant capacity of the sample to protect the fluorescent protein fluorescein from damage by the hypochlorite radical which is generated from sodium hypochlorite.

Thus, in one aspect, a collagen composition having an undenatured type II collagen according to the present disclosure may have a total ORAC of about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 600 μmol TE/g or greater, such as about 700 μmol TE/g or greater, such as about 750 μmol TE/g or greater, such as about 800 μmol TE/g or greater, such as about 825 μmol TE/g or greater, up to about 1000 μmol TE/g, or any ranges or values therebetween.

Furthermore, in one aspect, a collagen composition having an undenatured type II collagen according to the present disclosure may have a ORAC against peroxyl radicals of about 1 μmol TE/g or greater, such as about 2.5 μmol TE/g or greater, such as about 5 μmol TE/g or greater, such as about 7.5 μmol TE/g or greater, such as about 10 μmol TE/g or greater, such as up to about 10.5 μmol TE/g or greater, up to about 50 μmol TE/g, or any ranges or values therebetween.

Similarly, in one aspect, a collagen composition having an undenatured type II collagen according to the present disclosure may have a ORAC against hydroxyl radicals of about 10 μmol TE/g or greater, such as about 15 μmol TE/g or greater, such as about 20 μmol TE/g or greater, such as about 25 μmol TE/g or greater, such as about 27.5 μmol TE/g or greater, such as about 30 μmol TE/g or greater, up to about 40 μmol TE/g, or any ranges or values therebetween.

Additionally or alternatively, in one aspect, a collagen composition having an undenatured type II collagen according to the present disclosure may have a ORAC against peroxynitrite of about 0.5 μmol TE/g or greater, such as about 1 μmol TE/g or greater, such as about 1.5 μmol TE/g or greater, such as about 2 μmol TE/g or greater, such as about 2.25 μmol TE/g or greater, up to about 5 μmol TE/g, or any ranges or values therebetween.

In one aspect, a collagen composition having an undenatured type II collagen according to the present disclosure may have a ORAC against singlet oxygen of about 500 μmol TE/g or greater, such as about 550 μmol TE/g or greater, such as about 600 μmol TE/g or greater, such as about 650 μmol TE/g or greater, such as about 700 μmol TE/g or greater, such as about 725 μmol TE/g or greater, up to about 1000 μmol TE/g, or any ranges or values therebetween.

Furthermore, in one aspect, a collagen composition having an undenatured type II collagen according to the present disclosure may have a ORAC against hypochlorite of about 25 μmol TE/g or greater, such as about 30 μmol TE/g or greater, such as about 35 μmol TE/g or greater, such as about 40 μmol TE/g or greater, such as about 45 μmol TE/g or greater, such as up to about 50 μmol TE/g or greater, up to about 75 μmol TE/g, or any ranges or values therebetween.

Furthermore, as discussed above, the collagen composition is combined with a SOD enriched supplement. In one aspect, the SOD enriched supplement may be a SOD enriched marine phytoplankton, and may therefore be natural, non-GMO, and vegan. In one such aspect, the marine phytoplankton may be enriched with SOD by culturing under abiotic stress, or any other method known in the art sufficient to yield a SOD activity as described above. In a further aspect, the SOD enriched phytoplankton may be of the species Tetraselmis chuii, which has been found to be susceptible to high levels of SOD enrichment. Thus, in one aspect, the SOD enriched supplement is a biomass of T. chuii enriched with SOD, or an extract of the SOD enriched T. chuii, such as a protein extract, or a combination thereof. For instance, in one such aspect, the SOD enriched T. chuii may be available under the trade name Oceanix™. However, in a further aspect, the SOD enriched supplement may be a blend of marine phytoplankton, where at least a portion of the blend are T. chuii.

In one aspect, the present disclosure has found that a superoxide dismutase (SOD) enriched supplement containing a SOD activity of about 500 IU per administration or more, such as about 525 IU or more, such as about 550 IU or more, such as about 575 IU or more, such as about 600 IU or more, such as about 625 IU or more, such as about 650 IU or more, such as about 675 IU or more, such as about 700 IU or more per administration, such as about 725 IU or more, such as about 750 IU or more, such as about 775 IU or more, such as about 800 IU or more, such as about 825 IU or more, such as about 850 IU or more, such as about 875 IU or more, such as about 900 IU or more, such as about 925 IU or more, such as about 950 IU or more, such as about 975 IU or more, such as about 1000 IU or more, such as about 1025 IU or more, such as about 1050 IU or more, such as up to about 2000 IU or less per administration, can improve joint and muscle health and recovery in healthy mammals. Thus, in one aspect, the SOD enriched supplement is contained in the composition at an activity level amount sufficient to improve improves the fitness, measured as the time to exhaustion, reduce joint and/or muscle pain during intensive physical activity in healthy mammal, lengthen the period of physical activity or intensive physical activity in a healthy mammal before joint pain and/or muscle pain is experienced, or a combination thereof.

Furthermore, in one aspect, the SOD enriched supplement may have an SOD activity of about 15,000 IU/g or greater, such as about 20,000 IU/g or greater, such as about 25,000 IU/g or greater, such as about 30,000 IU/g or greater, such as about 35,000 IU/g or greater, such as about 40,000 IU/g or greater, such as about 42,500 IU/g or greater, up to a SOD activity of about 55,000 IU/g or less.

In one aspect, the SOD enriched supplement according to the present disclosure may have a total ORAC of about 100 μmol TE/g or greater, such as about 150 μmol TE/g or greater, such as about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 565 μmol TE/g or greater, up to about 750 μmol TE/g, or any ranges or values therebetween.

Furthermore, in one aspect, the SOD enriched supplement according to the present disclosure may have a ORAC against peroxyl radicals of about 10 μmol TE/g or greater, such as about 15 μmol TE/g or greater, such as about 20 μmol TE/g or greater, such as about 25 μmol TE/g or greater, such as about 30 μmol TE/g or greater, up to about 50 μmol TE/g, or any ranges or values therebetween.

Similarly, in one aspect, the SOD enriched supplement according to the present disclosure may have a ORAC against hydroxyl radicals of about 50 μmol TE/g or greater, such as about 75 μmol TE/g or greater, such as about 100 μmol TE/g or greater, such as about 125 μmol TE/g or greater, such as about 150 μmol TE/g or greater, such as about 175 μmol TE/g or greater, up to about 250 μmol TE/g, or any ranges or values therebetween.

Additionally or alternatively, in one aspect, the SOD enriched supplement according to the present disclosure may have a ORAC against peroxynitrite of about 1 μmol TE/g or greater, such as about 2.5 μmol TE/g or greater, such as about 5 μmol TE/g or greater, such as about 7.5 μmol TE/g or greater, such as about 8 μmol TE/g or greater, up to about 15 μmol TE/g, or any ranges or values therebetween.

In one aspect, the SOD enriched supplement according to the present disclosure may have a ORAC against superoxide anion of about 150 μmol TE/g or greater, such as about 200 μmol TE/g or greater, such as about 250 μmol TE/g or greater, such as about 300 μmol TE/g or greater, such as about 325 μmol TE/g or greater, such as about 350 μmol TE/g or greater, up to about 450 μmol TE/g, or any ranges or values therebetween.

Thus, in one aspect, the SOD enriched supplement may be present in the composition such that the composition has a total ORAC capacity from the SOD enriched supplement of about 2.5 μmol TE or greater, such as about 3 μmol TE or greater, such as about 3.5 μmol TE or greater, such as about 4 μmol TE or greater, such as about 4.5 μmol TE or greater such as about 5 μmol TE or greater, such as about 5.5 μmol TE or greater, such as about 6 μmol TE or greater, such as about 6.5 μmol TE or greater, such as about 7 μmol TE or greater, such as about 7.5 μmol TE or greater, such as about 8 μmol TE or greater, such as about 8.5 μmol TE or greater, such as about 9 μmol TE or greater, such as about 9.5 μmol TE or greater, such as about 10 μmol TE or greater, such as about 10.5 μmol TE or greater, such as about 11 μmol TE or greater, such as about 11.5 μmol TE or greater, such as about 12 μmol TE or greater, such as about 12.5 μmol TE or greater, such as about 13 μmol TE or greater, such as about 13.5 μmol TE or greater, such as about 14 μmol TE or greater, such as about 20 μmol TE or less.

For instance, in one aspect, the SOD enriched supplement may be present in the composition in an amount of about 1 mg to about 100 mg, such as about 5 mg to about 75 mg, such as about 10 mg to about 50 mg, such as about 15 mg to about 40 mg, such as about 20 mg to about 30 mg, or any ranges or values therebetween. Furthermore, in one aspect, the SOD enriched supplement may be present in the composition in an amount of about 10% to about 70% by weight, such as about 15% to about 65%, such as about 20% to about 60%, such as about 25% to about 55%, such as about 30% to about 50%, such as about 33% to about 45% by weight, based upon the total weight of the composition.

Thus, in one aspect, the collagen composition and the SOD enriched supplement may be present in the composition in a ratio of collagen composition to SOD enriched supplement of about 100:1 to about 1:100, such as about 10:1 to about 1:10, such as about 5:1 to about 1:5, such as about 2:1 to about 1:2, such as about 1.6:1 to about 1:1, or any ranges or values therebetween.

Furthermore, the SOD enriched supplement, and thus, the composition, may also include various other components that provide a further benefit to the healthy mammal.

For instance, the SOD enriched supplement and/or the composition may contain one or more vitamins, minerals, essential fatty acids, amino acids, polyphenols stilbenoids, curcumininoids, tannins, flavones, flavonols, flavan-3-ols, flavanones, anthocyanidins, anthocyanins, isoflavones, flavanonols, proanthocyanidins, dihydroxybenzoic acids, carotenoids, and pyridine alkaloids. In one aspect, the composition may contain an effective amount of an antioxidant, an amino acid, an essential fatty acid, a polyphenol, or combinations thereof.

For example, in one aspect the SOD enriched supplement and/or the composition contains at least one vitamin, such as at least one of vitamin B, vitamin C, and vitamin E. Vitamins may be contained in the SOD enriched supplement and/or the composition in an amount of from about 50 μg/g of supplement to about 5000 μg/g, such as about 100 μg/g to about 4500, such as about 250 μg/g to about 4000 μg/g, such as about 400 μg/g to about 3500 μg/g, or any ranges or values therebetween. The above ranges may be for any one vitamin alone or a total amount of all vitamins. In one aspect, vitamin E is present in the SOD enriched supplement or composition in an amount of about 100 μg/g to about 1000 μg/g, such as about 250 μg/g to about 750 μg/g, such as about 400 μg/g to about 600 μg/g, or any ranges or values therebetween. In another aspect, vitamin C is present in the SOD enriched supplement or the composition in an amount of about 1000 μg/g to about 5000 μg/g, such as about 2000 μg/g to about 4000 μg/g, such as about 3000 μg/g to about 3750 μg/g, or any ranges or values therebetween.

Furthermore, in an aspect, the SOD enriched supplement or the composition contains at least one mineral, such as at least one of potassium magnesium, zinc, or calcium. Minerals may be contained in the SOD enriched supplement or the composition in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the SOD enriched supplement or composition contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the SOD enriched supplement or the composition contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the SOD enriched supplement or the composition contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

Additionally, the SOD enriched supplement may further include at least one additive that enhances sports performance or that further contributes to reducing oxidative stress. For instance, in one aspect, an additive may be one or more of curcumin, spirulina, astaxanthin, or other carotenoids. Furthermore, in one aspect, the present disclosure may include one or more microalgae with high SOD and ORAC levels, where the one or more microalgae are different than a microalgae selected as the SOD enriched supplement. Particularly, such microalgae may further help to reduce oxidative stress, and may contribute further anti-inflammatory properties and protection against infections, including improvement in immune health. Moreover, in one aspect, an additive may include one or more probiotics.

It should be understood that any further components may be additives to the composition, or may be contained in the SOD enriched supplement. For instance, in one aspect, a phytoplankton is selected and/or enriched to provide one or more vitamins and/or minerals in addition to the SOD activity discussed herein.

Furthermore, the present disclosure has found that it may also be beneficial if the SOD enriched supplement has a structure containing a high percentage of protein. Thus, in one aspect, the SOD enriched supplement is about 5% to about 75% protein by weight of the supplement, such as about 10% to about 70%, such as about 15% to about 65%, such as about 20% to about 60%, such as about 25% to about 55%, such as about 30% to about 50% protein by weight, or any ranges or values therebetween.

In one aspect, the composition is administered once per day, twice per day, three times per day, up to about five times per day. However, in one aspect, the SOD enriched supplement is administered once per day.

Notwithstanding the final amounts of collagen composition, SOD enriched supplement, additives contained in the composition, or delivery method used, in one aspect, a mammal being administered the composition may exhibit about 10% or greater fitness than a mammal not being administered the composition, as measured based upon increased time to exhaustion, which will be discussed in greater detail in the examples, such as about 15% or greater, such as about 20% or greater, such as about 25% or greater, such as about 30% or greater, such as about 35% or greater fitness than a mammal not being administered the composition.

Furthermore, in one aspect, a mammal being administered the composition while undergoing physical activity or intensive physical activity according to the present disclosure may exhibit a lessening of cholesterol of about 10% greater than a mammal not being administered the composition, such as about 15% greater, such as about 20% greater, such as about 30% greater, such as about 40% greater, such as about 50% greater, such as about 60% greater, such as about 70% greater, such as about 80% greater, such as about 90% greater, such as about 100% greater that a mammal not being administered the composition, even if the mammal is undergoing a similar or identical exercise regime.

In one aspect, a mammal being administered the composition while undergoing physical activity or intensive physical activity according to the present disclosure may have about 10% less oxidative stress markers, indicating less muscle damage and/or soreness, than a mammal not undergoing administration according to the present disclosure, such as about 15% less, such as about 20% less, such as about 25% less, such as about 30% less oxidative stress markers than a mammal not being administered the composition, even if the mammal is undergoing a similar or identical exercise regime.

Similarly, in one aspect, a mammal being administered the composition while undergoing physical activity or intensive physical activity according to the present disclosure may have about 10% less lactate, indicating less muscle damage and/or sureness, than a mammal not undergoing administration according to the present disclosure, such as about 15% less, such as about 20% less, such as about 25% less, such as about 30% less, such as about 35% less, such as about 40% less lactate than a mammal not being administered the composition that is undergoing a similar or identical exercise regime.

Furthermore, in one aspect, a mammal being administered the composition while undergoing physical activity or intensive physical activity according to the present disclosure may have a concentration of cartilage breakdown indicators, such as cartilage oligomeric matrix protein (COMP), indicating less joint damage, cartilage damage, and/or pain, of about 5% less than a mammal not being administered the composition according to the present disclosure, such as about 10% less, such as about 15% less, such as about 20% less, such as about 25% less cartilage breakdown indicators as compared to a mammal not being administered the composition, even if the mammal is undergoing a similar or identical exercise regime.

Additionally or alternatively, in an aspect, a mammal being administered the composition while undergoing physical activity or intensive physical activity according to the present disclosure may have a concentration of bone growth (or regrowth) indicators, such as osteocalcin, of about 5% or greater than a mammal not being administered the composition according to the present disclosure, such as about 10% or greater, such as about 15% or greater, such as about 20% or greater concentration of bone growth indicators than a mammal not being administered the composition, even if the mammal is undergoing a similar or identical exercise regime.

While various aspects and benefits have been discussed, in one aspect, the composition is incorporated into a suitable delivery form prior to incorporation into a dosage form as discussed below. In one aspect, the composition may be coated onto a seed in order to form a multi-particulate dosage form that may be used in one or more of the administrations discussed in greater detail below. For instance, these multi-particulates may contain a starter particle or pellet, also known as seeds, beads, nonpareils, micro-granules, or starter cores. The cores are formed of inert material such as sucrose or microcrystalline cellulose. These cores are used as starter material and layered with active ingredient compounds before being incorporated into a final oral dosage formulation.

Nonetheless, in one aspect, the delivery form may include a lipid multiparticulate (LMP). For instance, such an aspect may include one or more particles, wherein each of the particles includes a core containing a lipid matrix having the composition dispersed therein, as the composition may be a fat-soluble active ingredient. The plurality of particles may also include one or more outer layers disposed thereon that may include one or more active ingredients, which may include the collagen composition or the SOD enriched supplement, or which may be a further active ingredient. The plurality of particles may be incorporated into a capsule or any other suitable oral or other dosage form discussed in greater detail below.

In one aspect utilizing a LMP delivery, the lipid matrix of the core may be formulated such that the core contains from about 10% to about 60% by volume of the composition. For example, in certain embodiments, the core may contain at least about 15% by volume of the composition, such as at least about 20% by volume, such as at least about 25% by volume, such as at least about 30% by volume, such as at least about 35% by volume, such as at least about 40% by volume, such as at least about 45% by volume, such as at least about 50% by volume, or any ranges or values therebetween.

In certain aspects, the lipid matrix of the core may include a) at least one low flow point excipient, b) at least one high flow point excipient, c) at least one low-flow point surfactant, and c) optionally an antioxidant.

The cores disclosed herein may include a lipid matrix that contains a low-flow point excipient. For example, in certain embodiments the lipid matrix may contain one or more low-flow point excipients. Low flow point excipients generally include fatty alcohols, fatty acids, fatty acid esters of glycols and poly glycols, fatty acid esters of polyglycerol and fatty acid esters of glycerol (glycerides) with flow points of less than 50° C. When the low flow point excipient is a relatively pure material, the melting point is also less than 50° C. A preferred class of low flow point excipients are low flow point glycerides. By “low flow point” excipient, such as a glyceride, is meant that the melting point of the excipient, such as a glyceride, is less than 50° C. In some embodiments, the low flow point glyceride has a melting point of less than 40° C. In some embodiments, the low-flow point excipient, such as glyceride, is a mixture of compounds, having a flow point of 50° C. or less. In some embodiments, the low-flow point excipient, such as glyceride, has a flow point of 40° C. or less. In some embodiments, the low-flow point glyceride has a low flow point of 30° C. or less. Exemplary low flow point glycerides include polyglycolized glycerides, such as some of the Gelucire products manufactured by Gattefosse, such as Gelucire® 43/01 having a nominal melting point of 43° C. Mixtures of low flow point glycerides are also effective, such as mixtures of Gelucire® 43/01 (010-018 triglycerides), Gelucire® 50/13 (stearoyl polyoxylglycerides), Gelucire® 44/14 (lauroyl macrogol-32 glycerides), and mixtures thereof. Other glycerides may also be used, such as fatty acid esters of glycols and poly glycols, and fatty acid esters of polyglycerols.

A function of the low flow point excipient is to ensure that at least a significant portion of the formulation matrix softens when ingested orally by a patient in need of therapy, at the temperature of the GI tract (about 37 for humans). This allows the formulation to break down by digestion in the gastro-intestinal (GI) tract, and ultimately to disperse in the GI tract to promote dissolution and absorption of the active. In certain embodiments the low flow point excipient provides a significant portion of the formulation matrix to be present in a non-crystalline liquid or amorphous state when ingested and softened in the GI tract.

Exemplary low flow point fatty alcohols include myristyl alcohol (Tm 38° C.), lauryl alcohol (Tm 23° C.). and capric alcohol (Tm 7° C.). Exemplary low flow point fatty acids include lauric acid (Tm 44° C.). and oleic acid (Tm 16° C.).

In certain aspects, the cores may include a lipid matrix including a high-flow point excipient. For example, in certain embodiments the lipid matrix may contain one or more high-flow point excipients. By “high flow point” excipient is meant an excipient that has a flow point 50° C. or more. High flow point excipients may also have a melting point above 50° C. High flow point excipients generally include fatty alcohols, fatty acids, fatty acid esters of glycols and poly glycols, fatty acid esters of polyglycerol, fatty acid esters of glycerol (glycerides), waxes, polar waxes and other materials with flow points of greater than 50. A preferred class of high flow point excipients are “high flow point glycerides”. By high flow point glyceride is meant that the flow point or melting point of the glyceride is 50° C. or more. In some embodiments, the high flow point glyceride has a melting point of 60° C. or more. In some embodiments, the high-melting point glyceride is a mixture of compounds, having a flow point of 50° C. or more. In some aspects, the high-flow point glyceride has a flow point of 60° C. or more. In some aspects, the high flow point glyceride has a flow point of 70° C. or more.

Exemplary high flow point glycerides include glycerol behenate, glycerol dibehenate, glycerol palmitate, hydrogenated castor oil, and mixtures thereof. Often, the high flow point glyceride is a mixture of compounds that are formulated into a product and sold under a variety of trade names. Furthermore, exemplary high flow point and high melt point fatty alcohols include stearyl alcohol (Tm 58° C.). and behenyl alcohol (Tm 71° C.). Exemplary high flow point and high melt point fatty acids include palmitic acid (Tm 63° C.) and stearic acid (Tm>70° C.). Additionally or alternatively, exemplary waxes include paraffin wax, beeswax, candelilla wax, carnauba wax, and mixtures thereof.

A function of the high flow point excipient is to aid in the manufacturability of the cores by enabling the cores to congeal at a lower temperature to obtain solid particles during the melt-spray-congeal processing. In certain aspects the high flow point excipient aids the physical stability of the core formulation. In most embodiments, the high flow point excipient is not appreciably digested in the GI tract.

In some aspects, the cores or the lipid matrix of the cores may include other excipients to improve the performance and chemical stability of the formulations. In one aspect, a dispersing agent is included in the core. Exemplary dispersing agents include lecithin, glycerin monostearate, ethylene glycol palmitostearate, aluminum oxide, polyethylene alky ethers, sorbitan esters, and mixtures thereof. In one aspect, the cores include an antioxidant to maintain chemical stability of the active agent. Exemplary antioxidants include vitamin E, tocopheryl polyethylene glycol succinate (TPGS), rosemary extract, ascorbic acid, asorbyl palmitate, butylated hydroxyanisole (BHA), buytlated hydroxytoluene (BHT), and mixtures and combinations thereof.

Furthermore, in one aspect, a flow aid is used to improve the flow properties of the cores. Exemplary flow aids also known as glidants include calcium silicate, cab-o-sil, silicon dioxide, calcium phosphate tribasic, colloidal silicone dioxide, magnesium silicate, magnesium trisilicate, starch, talc, and other flow aids.

The cores described herein are generally a plurality of particles or beadlets that are solid at ambient temperature and are generally spherical in shape. By generally spherical is meant that while most particles are essentially spherical, they do not necessarily form “perfect” spheres. Such particle variations in spherical shapes are known to those persons of ordinary skill in the art of melt-spray-congeal processing and similar particulate forming methods.

The cores may have a size ranging from a mean diameter of about 40 μm to about 3000 μm, such as from about 50 μm to about 2500 μm, such as from about 80 μm to about 2000 μm, such as from about 100 μm to about 1500 μm, such as from about 200 μm to about 1000 μm, such as from about 300 μm to about 800 μm. To measure the diameters of the particulates, there are several methods that can be used, including laser diffraction, optical microscopy, and/or SEM.

In certain aspects, the cores containing the active ingredient and lipid matrix have a flow point above 25° C., such as above 30° C., such as above 35° C., such as above 40° C.

In one aspect, the lipid matrix may contain fatty alcohols, fatty acids, fatty acid esters of glycerol, glycols and poly glycols, fatty acid esters of polyglycerol, polyglycolized glycerides, C8-C18 triglycerides, stearoyl polyoxylglycerides, lauroyl macrogol-32 glycerides, caprylocaproyl macrogol-8 glycerides, oleoyl macrogol-6 glycerides, linoleoyl macrogol-6 glycerides, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, propylene glycol monocaprylate esters, propylene glycol dicaprate/dicaprylate esters, propylene glycol heptanoate, propylene glycol monostearate, propoylene glycol monooleate, propylene glycol monopalmitate, propylene glycol monomyristate, esterified alpha-tocopheryl polyethylene glycol succinate, propylene glycol monolaurate esters, polyoxyl 35 castor oil, polyoxyl 40 hydrogenated castor oil, lecithins, vitamin E, tocopheryl polyethylene glycol succinate (TPGS), sugar fatty acid esters, sorbitan fatty acid esters, polyoxyethylene sorbitan fatty acid esters, polyoxyethylene-polyoxypropylene copolymers, propylene glycol, triacetin, isorpropyl myristate, diethylene glycol monoethyl ether, polyethylene glycol, glycerol, rosemary extract, ascorbic acid, ascorbyl palmitate, butylated hydroxyanisole (BHA), butylated hydroxytoluene (BHT), and mixtures and combinations thereof.

In a further aspect, the lipid matrix composition comprises greater than 50 wt % of the low flow point excipient. In one aspect, the lipid matrix composition comprises at least 2 wt % of the high flow point excipient. In yet a further aspect, the lipid matrix composition comprises less than 30 wt % of the high flow point excipient. Moreover, in one aspect, the mass ratio of the low flow excipient to the high flow excipient is at least 20:1, such as at least 15:1, such as at least 10:1, such as at least 4:1, such as at least 3:1, such as at least 2:1, or any ranges or values therebetween.

In one aspect, the composition is contained within a lipid matrix containing stearyl alcohol, stearic acid, candelilla wax, and lecithin. In one aspect the lipid matrix may contain from about 40% about 60% by weight of the active ingredient, such as about 50% by weight of the active ingredient. Furthermore, in an aspect, the lipid matrix may contain from about 15% to about 25% by weight of stearyl alcohol, such as about 17% by weight of stearyl alcohol. Additionally or alternatively, in an aspect, the lipid matrix may contain from about 10% to about 20% by weight of stearic acid, such as from about 15% by weight of stearic acid. In one aspect, the lipid matrix may contain from about 10% to about 20% of a suitable wax, such as candelilla wax, such as from about 15% by weight of a suitable wax. Moreover, in one aspect, the lipid matrix may contain from about 1% to about 3% of a lecithin, such as about 2% of a lecithin, such as soy lecithin.

In certain aspects, the lipid matrix containing the active ingredient may be formed and then formulated into one or more particles having a generally spherical shape and a mean diameter ranging from about ranging from 40 μm to 3000 μm, such as from about 100 μm to 2000 μm, such as from about 300 μm to 1000 μm.

In one aspect, the lipid matrix is comprised of at least 10 wt % to 50 wt % of the low flow point excipient. In another aspect, the lipid matrix is comprised of at least 50 wt % to 75 wt % of the low flow point excipient.

In one aspect, the lipid matrix comprises at least 2 wt % of the high flow point excipient. In another aspect, the lipid matrix is comprised of about 1 wt % to about 30 wt % of the high flow point excipient, such as about 2 wt % to about 20 wt % of the high flow point excipient, such as about 3 wt % to about 15 wt % of the high flow point excipient.

The lipid matrix may also include a dispersing agent. In one aspect, the lipid matrix includes from about 0 wt % to about 20 wt %, such as from about 0.01 wt % to about 20 wt %, of a dispersing agent. In another aspect, the lipid matrix includes from about 2 wt % to about 10 wt % of a dispersing agent.

The lipid matrix may also include an antioxidant. In one aspect, the lipid matrix includes from about 0 wt % to about 20 wt %, such as from about 0.01 wt % to about 20 wt %, of an antioxidant. In one aspect, the lipid matrix comprise from about 1 wt % to about 15 wt % of an antioxidant.

The lipid matrix may also include a flow aid. In one aspect, the lipid matrix includes from about 0 wt % to about 5 wt %, such as from about 0.01 wt % to about 5 wt %, of a flow aid. In another aspect, the lipid matrix includes from about 0.5 wt % to about 2 wt % of a flow aid.

In certain aspects, the cores disclosed herein may be subjected to additional processing in order to deposit one or more layers of an active ingredient material on the cores disclosed herein. For example, the cores described herein may contain one or more outer layers disposed on the core that include at least one active ingredient, which may be the same as one or more components in the composition or different from the components in the composition. Further, the one or more outer layers disposed on the core can include an adhesive in addition to one or more active ingredients. For example, to provide the outer layer disposed on the core, the core can be coated with suitable adhesives and then further coated with active ingredients in order to provide a particle having one or more outer layers including one or more active ingredients surrounding the core.

In certain aspects, the one or more particles may be subjected to any suitable coating process for edible tablets or particles in order to apply an active ingredient to the core or to the particles disclosed herein. Suitable processes may include those know generally as the Wurster process. Wurster processes are known in the art and may be synonymous with certain fluid bed microencapsulation processes. Descriptions of Wurster processes are disclosed in U.S. Pat. Nos. 2,648,609 and 3,241,520. Generally, during the Wurster process, the cores or particles are placed in a bed, such as a fluidized bed. The fluidized bed uses differential air flow to create a cyclic movement of the particle material. The one or more cores can be placed in the particulate bed and different air streams can move the bed of particulate material (i.e. the cores) as they are coated with a material, such as a suitable adhesive or active ingredient material, in order to coat the core structure. Generally, the process can be continued until the desired thickness of active ingredient layer is achieved on the core. The particles disclosed herein may be subjected to a suitable Wurster process in order to provide one or more active ingredient layers to the particle. In some embodiments, the cores or particles disclosed herein may be coated via any suitable fluid bed coating process.

In some aspects, the cores and/or particles may be coated via any known drum coating process. Generally, a drum coating process refers to a method for coating particles via placing the particles inside a rotating drum and applying the desired coating material to the particle while rotating the particles within the drum. Devices suitable for drum coating particles are known and include those described in U.S. patent publication no. 2015/0144058. In certain embodiments the cores disclosed herein may be placed in any suitable drum coating device and an adhesive can be applied to the cores or particles. Once sufficiently coated with the adhesive, an active ingredient can then be applied to the cores or particles in the drum to create an active ingredient layer on the cores or particles. This process of applying a suitable adhesive and active ingredient can be repeated as many times as necessary in order to produce the desired number of active ingredient layers on the particles. In certain embodiments, the adhesive may be applied to the core or particle directly with one or more active ingredients.

In one aspect, the cores or particles may be coated with a suitable adhesive. Suitable adhesives may include pharmaceutical grade shellacs such as pharmaceutical glaze, which is an alcohol-based solution that can include various types of food-grade shellac. In certain embodiment, the pharmaceutical glaze may contain from about 20% by weight to about 51% by weight of shellac in an ethyl alcohol solution. The pharmaceutical glaze may further contain additional additives such as waxes, titanium dioxide, and combinations thereof. In certain embodiments, the pharmaceutical glaze utilized according to embodiments herein is certified as generally recognized as safe (GRAS) by the U.S. Food and Drug Administration (FDA). In certain embodiments, the adhesive may include a suitable non-animal-based product, such as zein. Zein generally refers to a class of prolamine protein found in corn that can be manufactured as an adhesive coating or binder.

Accordingly, in some aspects, the outer surface of the cores described herein are coated with a sufficient amount of a suitable adhesive to enable binding of the active ingredient to the outer surface of the core. Generally, the adhesive can be applied such that outer surface of the core becomes tacky or sticky, but is not so tacky such that the cores or particles agglomerate together. Once the adhesive is applied, an active ingredient can be applied to the particle to create an active ingredient layer thereon. In some embodiments, the process of layering the particle with an adhesive layer and layering the particle with an active ingredient can be repeated as many times as desired in order to form a particle having a desired amount of active ingredient layers. For example, the particles disclosed herein may include a core having at least one outer layer thereon containing at least one active ingredient, such as at least two outer layers, such as at least three outer layers, such as at least four outer layers, such as at least five outer layers, such as at least six outer layers, etc. In certain embodiments, each of the outer layers may include one or more active ingredients. In some embodiments, one or more of the outer layers may include the same active ingredient or a different active ingredient from the active ingredient(s) contained within the core. In certain embodiments, the particle may contain one or more outer layers that do not contain an active ingredient. For example, the particle may contain one or more outer layers comprised of a suitable coating material for sealing the particle or for providing a particular release profile upon ingestion.

In some embodiments, the one or more particles provided herein may be formulated into any suitable dosage formulation. For example, in certain aspects, the one or more particles provided herein may be placed into a capsule for delivery by oral ingestion. Exemplary capsules include hard gelatin capsules, soft gelatin capsules, HPMC capsules, as well as capsules made from other materials. The one or more particles may be suspended in an aqueous-based matrix or an oil-based matrix within the capsule itself. In certain embodiments where the particles are suspended in an aqueous-based matrix or an oil-based matrix, the aqueous-based matrix or oil-based matrix may additional include one or more active ingredients. In certain embodiments, the one or more particles may be contained within a monolithic enteric capsule suitable for providing a modified release profile when ingested.

Capsules normally include a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenans) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. See, e.g., U.S. Pat. Nos. 5,264,223, 5,756,123, and 5,756,123. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. See, e.g., EP 0401832, U.S. Pat. Nos. 3,493,407, 4,001,211, and 3,617,588, GB 1310697, and WO 2008/050209. The aforementioned manufacturing processes involve the use of solutions of the different ingredients that are needed for the making the telescoping fit hard capsule shells.

Hard capsules may be filled with active ingredients, such as the composition described herein, via procedures known in the art. Typically, active ingredients (e.g. SOD enriched supplement and type II collagen) are combined with various compatible excipients for ease of fill. The resulting fill may be a dry powder, a granulation, particles, lipid particles, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets. For example, in some embodiments, provided is a pharmaceutical composition that contains a capsule filled with the one or more particles disclosed herein. In some embodiments, the one or more particles have not been enterically coated for modified release or gastric protection.

Additionally or alternatively, in one aspect, the composition of the present disclosure may be included as an oil-in-water emulsion as a delivery form. Particularly, in one aspect, such an arrangement may allow one or more oil-soluble and/or one or more water-soluble active ingredients to be contained in the same delivery form. Alternatively, only oil-soluble components may be used (e.g. the SOD enriched supplement and the Type II collagen), and the emulsion may be used to incorporate the composition into a water-based application.

Nonetheless, the oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible. In some embodiments, the gum arabic may be comprised of a 100% modified gum arabic, such as Ticamulsion® A-2010 gum arabic powder. In certain embodiments, the gum arabic may be a mixture or blend of gum arabic and modified gum arabic. For example, in certain embodiments, the gum arabic may comprise Ticamulsion® 3020.

In certain aspects, the oil-in-water emulsion contains from about 10% to about 30% by weight of gum arabic. In some embodiments, the oil-in-water emulsion contains from about 15% to about 25% by weight of gum arabic. In some embodiments, the oil-in-water emulsion contains less than about 20% by weight of gum arabic, such as less than 15%, such as less than 10%, such at less than 5%.

The oil-in-water emulsion may also contain water. In certain aspects, the oil-in-water emulsion contains deionized water. Still, in certain aspects, the oil-in-water emulsion may contain any water suitable for human ingestion and incorporation into dietary supplements designed for human ingestion.

The amount of water incorporated into the oil-in-water emulsion can vary depending on the desired hygroscopic and water-soluble ingredients that are incorporated into the oil-in-water emulsion. In certain aspects, the oil-in-water emulsion may contain from about 5% to 35% by weight of water. In some embodiments, the oil-in-water emulsion may contain from about 10% to about 30% by weight of water. In some embodiments, the oil-in-water emulsion may contain from about 15% to about 20% by weight of water. In some embodiments, the oil-in-water emulsion may contain less than about 20% by weight of water, such as less than about 15% by weight of water, such as less than about 10% by weight of water.

In certain aspects, the oil-in-water emulsions may have a water activity of less than about 0.6, such as less than about 0.55, such as less than about 0.5, such as less than about 0.45, such as less than about 0.40, such as less than about 0.35, such as less than about 0.30, such as less than about 0.25, such as less than about 0.20, such as less than about 0.15, such as less than about 0.10. Generally, water activity (a_{w}) described the equilibrium amount of water available for hydration of materials. Water activity generally measures the partial vapor pressure of water in the solution and divides that value by the partial vapor pressure of water at the same temperature to give a final reading. Water activity (a_{w}) can be measured according to the following:

a_{w}=p/p*

For instance, in one aspect, emulsions having water activities over 0.6 may lead to physical instability of the emulsions. Furthermore, it was discovered herein that emulsions having a water activity greater than 0.6 may cause disintegration or dissolve certain HPMC capsules.

In some aspects, the oil-in-water emulsion may contain one or more stabilizers or suspension promoting agents. For example, in certain aspects, the oil-in-water emulsion may contain one or more gum, such as gellan gum or xanthum gum. If included, the gellan gum or xanthum gum may be present in an amount of less than about 3.5% by weight of the oil-in-water emulsion, such as less than about 2.5% by weight, such as less than about 1.5% by weight, such as less than about 1.0% by weight, such as less than about 1.0% by weight.

In other aspects, the oil-in-water emulsion may contain one or more stabilizers such as silica. If included, silica may be present in an amount of less than about 2% by weight, such as less than about 1.5% by weight, such as less than about 1% by weight, such as less than about 0.5% by weight.

Furthermore, in one aspect, the oil-in-water emulsion may also contain one or more fat-soluble ingredients or nutrients. In certain aspects, the one or more fat-soluble ingredients or nutrients may be incorporated into the oil phase of the oil-in-water phase emulsion. Suitable fat-soluble ingredients include, but are not limited to retinol, vitamin E sourced from mixed tocopherols, beta carotene, ubiquinone, lecithin, sunflower lecithin, vitamin D, cannabinoids, hemp extracts, vitamin K, phosphatidyl choline, and combinations thereof.

In certain aspects, at least one or more fat-soluble ingredients may be incorporated in the oil-in-water emulsion in an amount of from about 0% by weight to about 50% by weight. For example, in some aspects, the oil-in-water emulsion contains less than about 50% by weight of one or more fat-soluble ingredients, such as less than about 40% by weight, such as less than about 30% by weight, such as less than about 20% by weight, such as less than about 10% by weight, such a less than about 5% by weight.

Moreover, in one aspect, the oil-in water emulsion may contain one or more additional antioxidants, in one or more of the water soluble phase, or the oil/fat soluble phase.

In some aspects, the oil-in-water emulsion disclosed herein may be provided in any suitable dosage form, such as capsules, tablets, gummy chewables, edible films, lozenges, powders, liquid suspensions, syrups, lipid micelles, spray-dried dispersions, nanoparticles, and the like. In one aspect, the dosage form is an oral dosage form such as a capsule. Capsules are well-known dosage forms that normally consist of a shell filled with one or more specific substances, i.e. the oil-in-water emulsion disclosed herein. The capsule shell may be a soft shell or a hard shell containing film-forming polymers, such as gelatin, modified starches, modified cellulosed, etc.

Furthermore, in an aspect, the oil-in-water emulsion may be formulated to be provided in a capsule. In certain aspects, the capsule may include any suitable HPMC-based capsule. Advantageously, the oral dosage form provided herein may include an HPMC-based capsule containing the oil-in-water emulsion disclosed herein. In one aspect, the HPMC-base capsule may include capsules manufactured by Lonza® Inc. under the tradename of Vcaps®, Vcaps® Plus, or DRcaps®. In some aspects, the capsule may include an modified HPMC-capsule that contains gellan gum and/or thalate. In certain embodiments, the capsule may include an HPMC capsule that has been treated so as to be acid resistant.

In one aspect, the capsule may contain a certain fill weight range or fill volume range. For example, in certain aspects, the capsule may be filled with from about 150 mg to about 1050 mg of the oil-in-water emulsion. In one aspect, the capsule may be filled with about 150 mg of the oil-in-water emulsion. Furthermore, in an aspect, the capsule may be filled with about 20 mg of the oil-in-water emulsion. Moreover, in an aspect, the capsule may be filled with about 280 mg of the oil-in-water emulsion. In some aspects, the capsule may be filled with about 380 mg or more of the oil-in-water emulsion, such as about 500 mg or more of the oil-in-water emulsion, such as about 700 mg or more of the oil-in-water emulsion, such as about 1,050 mg or more of the oil-in-water emulsion.

Alternatively, the oil-in-water emulsion may be contained in a nutritional product, such as a food product or in a beverage. For example, in certain aspects, the oil-in-water emulsion may be incorporated into a liquid nutritional product, such as a nutritional supplement or infant formula, to be consumed by a mammal. Furthermore, the oil-in-water emulsions provided herein may be added to any liquid nutritional product designed to provide nutritional supplementation to a mammal.

Nonetheless, in one aspect, the composition, either alone or as contained in one of the delivery methods above, can be administered orally as a solid, liquid, suspension, or gas. The composition may be administered via buccal or sublingual administration. In one embodiment, the composition may be administered as a capsule, tablet, caplet, pill, troche, drop, lozenge, powder, granule, syrup, tea, drink, thin film, seed, paste, herb, botanical, and the like.

Nonetheless, in addition to being administered orally, the composition can also be administered using other routes including intranasal, intravenous, intramuscular, intragastric, and the like.

When the composition, either alone or as part of a delivery form, is combined with food or beverage composition, the food or beverage composition may comprise any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, or the like.

The composition of the present disclosure may further comprise one or more excipients as further additives in the composition. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, and waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Ltd.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the pharmaceutical composition may comprise silica. For instance, the oxide particles may comprise precipitated silica particles. The silica particles may have a particle size (d50, laser defraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Once the above components are combined together to form the composition, the composition can optionally be combined with a coating material. In one embodiment, for instance, the composition may comprise a granular composition to which a coating material is applied that contains a fat. The coating material, for instance, may comprise a hydrogenated oil, such as hydrogenated palm oil. In one particular embodiment, the coating material may comprise hydrogenated palm oil combined with palm stearine. In one embodiment, the hydrogenated oil may be present in the pharmaceutical composition in an amount from about 5% to about 35% by weight. The palm stearine, on the other hand, may be present in the pharmaceutical composition in an amount from about 2% to about 10% by weight. When present together, the weight ratio between the hydrogenated palm oil and the palm stearine may be from about 10:1 to about 1:1, such as from about 6:1 to about 2:1. In one embodiment, the hydrogenated palm oil and the palm stearine are present at a weight ratio of about 4:1.

Furthermore, the mammal treated in accordance with the present disclosure can comprise any suitable mammal. For instance, the mammal may be human or canine. The composition can be fed to a mammal of any age such as from parturition through the adult life in the mammal. In various embodiments the mammal may be a human, dog, a cat, a horse, a pig, a sheep, or a cow. In many embodiments, the mammal can be in early to late adulthood. For instance, the active mammal may have an age that is at least 10%, such as least 15%, such as least 20%, such as least 25%, such as least 30%, such as least 35%, such as least 40%, such as least 45%, such as least 50%, such as least 55%, such as least 60%, such as least 65%, such as least 70%, such as least 75%, such as least 85%, such as least 90%, such as least 95% of its expected life span. The mammal may have an age such that it is less than about 95%, such as less than about 90%, such as less than about 85%, such as less than about 80%, such as less than about 75%, such as less than about 70%, such as less than about 65%, such as less than about 60%, such as less than about 55%, such as less than about 50%, such as less than about 45%, such as less than about 40%, such as less than about 35%, such as less than about 30%, such as less than about 25%, such as less than about 20%, such as less than about 15%, such as less than about 10% of its expected life span. A determination of life span may be based on actuarial tables, calculations, or the like.

Nonetheless, certain embodiments of the present disclosure may be better understood according to the following examples, which are intended to be non-limiting and exemplary in nature.

### Example 1

Female Wistar rats (8 weeks) randomly allocated into groups (n=7 in each) as follows:
Control
Exercise
Exercise+2.55 mg SOD enriched supplement (Oceanix™)+4 mg Undenatured Type II Collagen composition (UCII™) (for instance, 2.55 mg*d per rat is the equivalent of 25 mg Human Equivalent Dose (HED) per day for a human having a body weight of 60 kg)
Exercise+5.1 mg SOD enriched supplement (Oceanix™)+4 mg Undenatured Type II Collagen composition (UCII™) (for instance, 5.1 mg*d per rat is the equivalent of 50 mg HED per day for a human having a body weight of 60 kg)

Exercise Protocol: The exercise protocols performed on a motor-driven rodent treadmill (MAY-TME, Commat Limited, Ankara, Turkey). The treadmill included a stimulus grid at the back end of the treadmill which provided an electric shock if the animal placed its paw on the grid. The apparatus consisted of a 5-lane animal exerciser utilizing single belt construction with dividing walls suspended over the tread surface. All exercise tests performed during the same time period of the day to minimize diurnal effects. All rats pre-trained in order for the animals to be exposed to the treadmill equipment and handling for 1 week. The exercise training period based on (i) 1st day, 10 m/min, 10 min, (ii) 2nd day, 20 m/min, 10 min, (iii) 3rd day, 25 m/min, 10 min, (iv) 4th day, 25 m/min, 20 min and (v) 5th day, 25 m/min, 30 min. After 1-week treadmill familiarization to eliminate novel and stress effects, animals in treadmill exercise groups ran on the treadmill 25 m/min, 45 min/day and five days per week for 8 weeks according to the protocol published earlier (Sahin et al., 2016).

TABLE 1

| | Control | Exercise | Oceanix 1 + UCII | Oceanix 2 + UCII |
|---|---|---|---|---|
| Initial Body | 207.29 + 4.85 | 217.71 + 7.6 | 219.00 + 13.78 | 217.71 + 7.47 |
| Weight, g | | | | |
| Final Body | 321.57 + 4.08^{a} | 283.43 + 4.49b | 293.43 + 5.74^{b} | 291.00 + 5.47^{b} |
| Weight, g | | | | |
| Distance Run | — | 977.29 + 5.13 | 988.71 + 9.39 | 992.71 + 7.51 |
| Avg, min | | | | |
| Exhaustion | 41.29 + 2.63^{d} | 103.86 + 4.25^{c} | 128.57 + 3.44 | 134.14 + 4.34^{a} |
| Time, min | | | | |

Different symbols indicate statistical differences among the groups
Oceanix 1 + UCII: Ex + 2.55 mg Oceanix + 4 mg UCII (2.55 mg*d/rat = 25 mg HED for a 60 kg BW/d)
Oceanix 2 + UCII: Ex + 5.1 mg Oceanix + 4 mg UCII (5.1 mg*d/rat = 25 mg HED for a 60 kg BW/d)

Referring to Table 1, rats being administered a composition containing a undenatured type II collagen and a SOD enriched supplement exhibited a lower body weight as compared to the control, as well as improved time to exhaustion and average distance run as compared to the control and the exercise sample.

TABLE 2

| Markers | Control | Exercise | Oceanix 1 + UCII | Oceanix 2 + UCII |
|---|---|---|---|---|
| Glucose, | 108.29 + 3.19^{a} | 102.14 + 1.77^{ab} | 96.14 + 2.56^{b} | 96.29 + 2.29^{b} |
| mg/dL | | | | |
| Total | 98.51 + 1.66^{a} | 92.06 + 1.57^{ab} | 83.02 + 1.95^{bcd} | 75.12 + 2.16^{d} |
| Cholesterol, | | | | |
| mg/dL | | | | |
| Triglyceride, | 103.51 + 1.17^{b} | 96.58 + 1.74^{ab} | 90.59 + 3.46^{cd} | 83.26 + 2.5^{c} |
| mg/dL | | | | |
| Total | 6.56 + 0.19 | 6.59 + 0.11 | 6.48 + 0.09 | 6.49 + 0.11 |
| Protein, | | | | |
| g/dL | | | | |
| Albumin, | 3.43 + 0.10 | 3.51 + 0.09 | 3.50 + 0.05 | 3.46 + 0.11 |
| g/dL | | | | |
| Globulin, | 3.09 + 0.12 | 3.29 + 0.10 | 3.03 + 0.09 | 3.17 + 0.09 |
| g/dL | | | | |
| Alanine | 97.29 + 4.65 | 98.43 + 5.42 | 95.86 + 5.55 | 96.29 + 3.26 |
| Transaminase | | | | |
| (ALT), u/L | | | | |
| Aspartate | 118.43 + 6.21 | 116.86 + 6.28 | 113.00 + 6.50 | 112.43 + 4.81 |
| Transaminasse | | | | |
| (AST), u/L | | | | |
| Total | 0.24 + 0.01 | 0.24 + 0.01 | 0.23 + 0.01 | 0.24 + 0.01 |
| Bilirubin, | | | | |
| mg/dL | | | | |
| Creatinine | 125.80 + 1.75^{d} | 193.6 + 2.86^{a} | 157.98 + 3.08^{c} | 148.41 + 2.29^{c} |
| kinase | | | | |
| (CK), IU/L | | | | |
| Creatinine, | 0.48 + 0.03 | 0.47 + 0.02 | 0.47 + 0.03 | 0.46 + 0.03 |
| mg/dL | | | | |
| Blood Urea | 20.47 + 0.77 | 20.86 + 0.23 | 20.06 + 0.73 | 20.84 + 0.59 |
| Nitrogen | | | | |
| (BUN), mg/dL | | | | |

Different symbols indicate statistical differences among the groups
Oceanix 1 + UCII: Ex + 2.55 mg Oceanix + 4 mg UCII (2.55 mg*d/rat = 25 mg HED for a 60 kg BW/d)
Oceanix 2 + UCII: Ex + 5.1 mg Oceanix + 4 mg UCII (5.1 mg*d/rat = 25 mg HED for a 60 kg BW/d)

Referring to Table 2, rats being administered a composition containing a undenatured type II collagen and a SOD enriched supplement exhibited lower glucose, cholesterol, triglycerides, and creatine kinase as compared to the control and the exercise sample with no change in liver and kidney enzymes.

TABLE 3

| | | | Oceanix 1 + | Oceanix 2 + |
|---|---|---|---|---|
| Markers | Control | Exercise | UCII | UCII |
| Serum | 0.76 ± 0.02^{a} | 0.68 ± 0.01^{ab} | 0.54 ± 0.03^{cd} | 0.43 ± 0.01^{e} |
| Malondialdehyde | | | | |
| (MDA) μmol/L | | | | |
| Muscle MDA, | 1.91 ± 0.05^{a} | 1.67 ± 0.04^{b} | 1.39 ± 0.03^{de} | 1.17 ± 0.06^{f} |
| nmol/mg | | | | |
| Superoxide | 70.88 ± 1.57^{d} | 82.28 ± 1.82^{c} | 100.63 ± 2.62^{b} | 114.00 ± 2.91^{a} |
| Dismutase | | | | |
| (SOD), U/mL | | | | |
| Catalase | 140.83 ± 2.80^{d} | 153.00 ± 2.85^{c} | 166.71 ± 2.51^{ab} | 177.54 ± 1.84^{a} |
| (CAT), U/mL | | | | |
| Glutathione | 71.13 ± 1.97^{e} | 82.00 ± 2.61^{d} | 97.69 ± 2.62^{bc} | 108.28 ± 3.21^{a} |
| peroxidase | | | | |
| (GSHPx), U/ml | | | | |

Different symbols indicate statistical differences among the groups
Oceanix 1 + UCII: Ex + 2.55 mg Oceanix + 4 mg UCII (2.55 mg*d/rat = 25 mg HED for a 60 kg BW/d)
Oceanix 2 + UCII: Ex + 5.1 mg Oceanix + 4 mg UCII (5.1 mg*d/rat = 25 mg HED for a 60 kg BW/d)

Referring to Table 3, rats being administered a composition containing a undenatured type II collagen and a SOD enriched supplement exhibited lower oxidative stress markers (MDA) as well as increased antioxidant enzymes as compared to the control and the exercise sample.

TABLE 4

| | Groups | | | |
|---|---|---|---|---|
| Details | Control | Exercise | Oceanix 1 + UCII | Oceanix 2 + UCII |
| Lactate, | 2.80 + 0.07d | 5.76 + 0.12a | 3.82 + 0.08c | 3.39 + 0.05c |
| mmol/L | | | | |
| Osteocalcin, | 26.93 + 0.53d | 30.75 + 0.49d | 34.62 + 0.47bc | 37.71 + 0.65a |
| ng/mL | | | | |
| Myoglobin, | 21.95 + 0.65f | 42.34 + 0.87b | 30.51 + 0.81de | 27.44 + 0.72d |
| ng/mL | | | | |
| Cartilage | 6.86 + 0.21^{a} | 6.98 + 0.32a | 5.85 + 0.32ab | 5.23 + 0.18b |
| Oligomeric | | | | |
| Matrix Protein | | | | |
| (COMP), ng/mL | | | | |
| IL-1β, | 19.63 + 0.50^{a} | 19.89 + 0.63a | 18.22 + 0.81ab | 17.22 + 0.49ab |
| pg/mL | | | | |
| IL-6, | L | 9.06 + 0.45 | 7.77 + 0.37bc | 7.11 + 0.55c |
| pg/mL | | | | |
| Tumor Necrosis | 19.72 + 0.37a | 18.42 + 0.68ab | 15.86 + 0.31d | 14.70 + 0.35d |
| Factor Alpha | | | | |
| (TNFα), pg/mL | | | | |

Data are presented as mean and standard error
Means in the same line without a common superscript differ significantly (p < 0.05)

Referring to Table 4, rats being administered a composition containing a undenatured type II collagen and a SOD enriched supplement exhibited lower lactate, cytokines, and cartilage oligomeric matrix protein, as well as increased osteocalcin as compared to the control and the exercise sample.

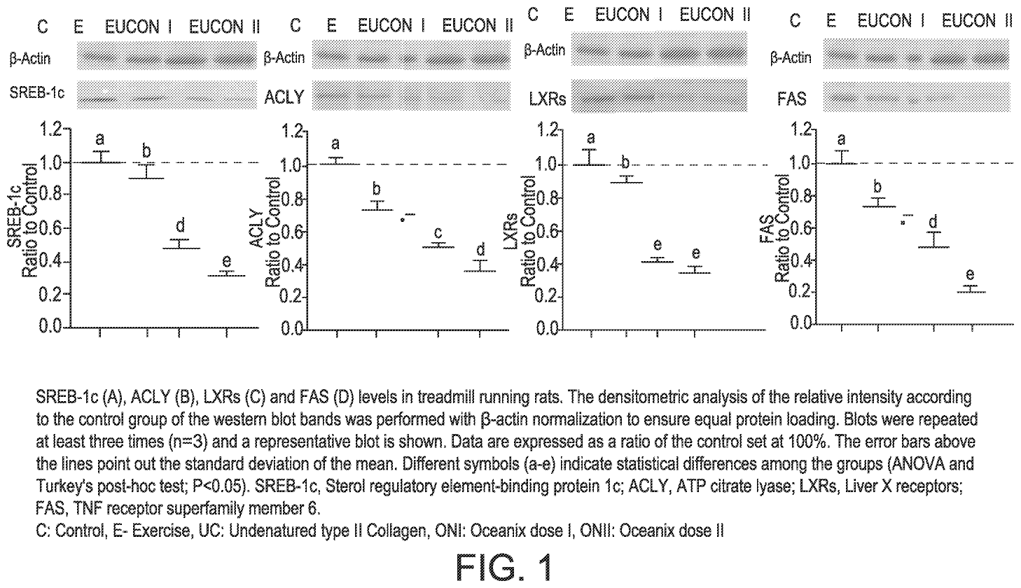

Furthermore, referring to FIG. 1, levels of SREB-1c, ACLY, LXRx, and FAS were tested in treadmill running rats. As shown, samples administered a composition according to the present disclosure exhibited lower levels in each of the test proteins, indicating lower cholesterol levels.

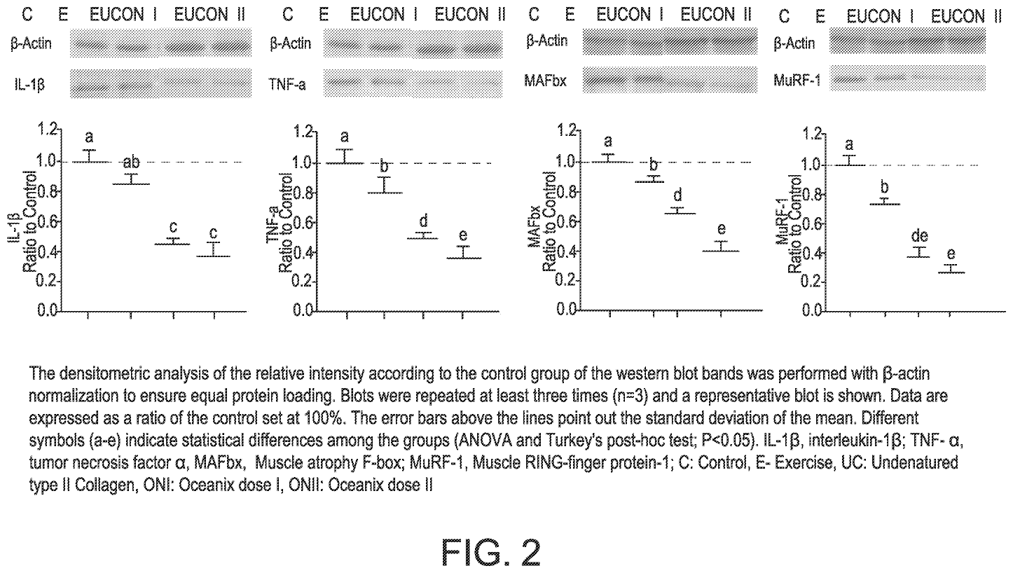

Referring to FIG. 2, levels of IL-113, TNF-α, MAFbx, and MuRF-1 were tested in treadmill running rats. As shown, samples administered a composition according to the present disclosure exhibited lower levels in each of the test proteins, indicating lower inflammation and muscle atrophy.

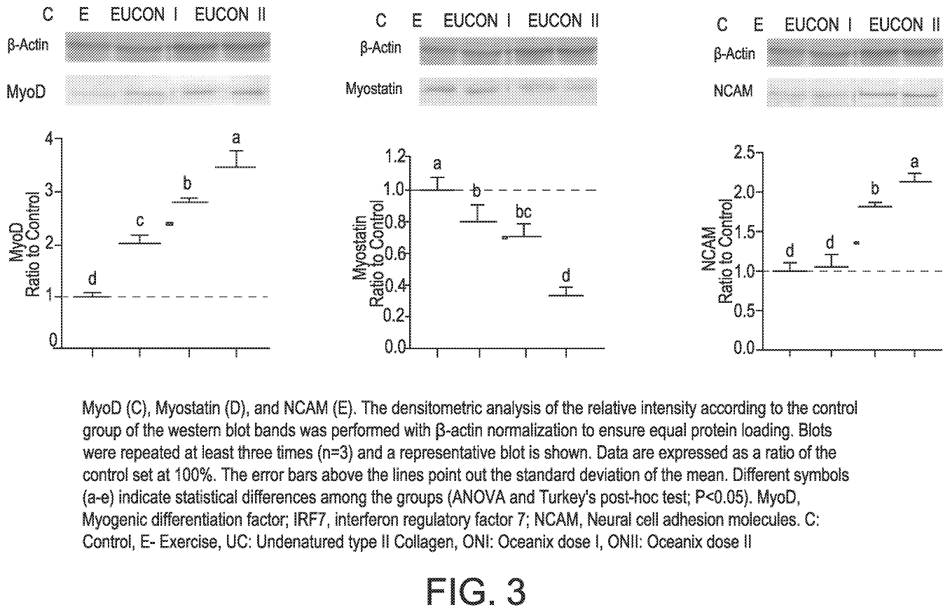

Finally, referring to FIG. 3, MYOD, IRF7 and NCAM were tested in treadmill running rats. As shown, MYOD was elevated compared to the controls, IRF7 was increased, and NCAM was decreased, indicating improved muscle differentiation.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A method of improving one or more of fitness, joint pain, or muscle pain in healthy mammals that have undergone or are undergoing physical activity, the method comprising
administering a composition comprising a collagen composition and a SOD enriched supplement to a healthy mammal in an amount sufficient to lessen joint pain, lessen muscle pain, or improve fitness of the mammal, and
wherein the collagen composition comprises a type II collagen, and wherein the type II collagen is present in the composition in an amount from about 1 mg to about 1000 mg.

2. The method of claim 1, wherein the SOD enriched supplement and the collagen composition are present in the composition at a ratio of type II collagen to SOD enriched supplement of about 100:1 to about 1:100.

3. The method of claim 1, wherein the SOD enriched supplement comprises T. chuii.

4. The method of claim 3, wherein the SOD enriched supplement is a biomass of T. chuii, an extract of T. chuii, or a combination thereof.

5. The method of claim 3, wherein the SOD enriched supplement is present in the composition in an amount of about 1 mg to about 100 mg.

6. The method of claim 1, wherein the SOD enriched supplement has a SOD activity in the composition of about 500 IU or greater.

7. The method of claim 1, wherein an oxygen radical absorbance capacity (ORAC) of the SOD enriched supplement is about 100 μmol TE/g or greater.

8. The method of claim 1, wherein an oxygen radical absorbance capacity (ORAC) of the SOD enriched supplement in the composition is about 2.5 μmol TE or greater.

9. The method of claim 1, wherein the composition is administered daily.

10. The method of claim 1, wherein the composition further includes at least one of a vitamin, a mineral, an essential fatty acid, or an amino acid.

11. The method of claim 10, wherein the SOD enriched supplement includes vitamin E, vitamin C, or a combination thereof.

12. The method of claim 10, wherein the SOD enriched supplement includes potassium, magnesium, calcium, or combinations thereof.

13. The method of claim 1, wherein the lessening of joint pain, muscle pain, or improvement in fitness is evidenced by an improvement in fitness of about 10% or greater than a mammal not being administered the composition, as measured by time to exhaustion.

14. The method of claim 1, wherein the lessening of joint pain, muscle pain, or improvement in fitness is evidenced by a lessening of cholesterol of about 10% greater than a mammal not being administered the composition.

15. The method of claim 1, wherein the lessening of joint pain, muscle pain, or improvement in fitness is evidenced by the mammal exhibiting about 10% less oxidative stress markers than a mammal not being administered the composition.

16. The method of claim 1, wherein the lessening of joint pain, muscle pain, or improvement in fitness is evidenced by the mammal exhibiting cartilage breakdown indicators of about 5% less than a mammal not being administered the composition.

17. The method of claim 1, wherein the healthy mammal is not suffering from age related decline.

18. The method of claim 1, wherein the healthy mammal is not suffering from arthritis.

19. The method of claim 1, wherein the physical activity is intense physical activity.

20. The method of claim 1, wherein the type II collagen is present in the composition in an amount of about 5 mg to about 500 mg.

21. The method of claim 20, wherein the type II collagen is present in the composition in an amount of about 15 mg to about 200 mg.

22. The method of claim 20, wherein the type II collagen is present in the composition in an amount of about 20 mg to about 100 mg.

23. The method of claim 1, wherein the mammal has an age of about 55% or less of its expected life span.

24. The method of claim 1, wherein the mammal has an age of about 60% or more of its expected life span.

25. The method of claim 1, wherein the type II collagen includes native type II collagen and undenatured type II collagen.

26. The method of claim 25, wherein the undenatured type II collagen is present in the composition in an amount of about 0.25 mg to about 100 mg.

27. A composition for improving one or more of fitness, joint pain, or muscle pain in healthy mammals that have undergone or are undergoing physical activity comprising,
a collagen composition comprising a type II collagen, the collagen composition having a total oxygen radical absorbance capacity (ORAC) of about 200 μmol TE/g or greater, wherein the type II collagen is present in the composition in an amount of from about 1 mg to about 1000 mg; and
a SOD enriched supplement having a total ORAC of about 100 μmol TE/g or greater.

28. The composition of claim 27, wherein the type II collagen includes native type II collagen and undenatured type II collagen.

29. The composition of claim 28, wherein the undenatured type II collagen is present in the composition in an amount of about 0.25 mg to about 100 mg.

30. The composition of claim 27, wherein the composition is formulated into one or more delivery forms.

31. The composition of claim 30, wherein the composition is coated on a core, contained in a lipid multiparticulate, or contained in an oil-in-water emulsion.

32. The composition of claim 30, wherein the one or more delivery forms is suitable for incorporation into a capsule, a beverage, a food product, or combinations thereof.

33. The composition of claim 27, wherein the SOD enriched supplement and the type II collagen are present in the composition at a ratio of type II collagen to SOD enriched supplement of about 100:1 to about 1:100.

34. The composition of claim 27, wherein the SOD enriched supplement comprises T. chuii.

35. The composition of claim 34, wherein the SOD enriched supplement is a biomass of T. chuii, an extract of T. chuii, or a combination thereof.

36. The composition of claim 27, wherein the SOD enriched supplement is present in the composition in an amount of about 1 mg to about 100 mg.

37. The composition of claim 27, wherein the wherein the SOD enriched supplement has a SOD activity in the composition of about 500 IU or greater.
